(19) United States
(12) Patent Application Publication
(10) Pub. No.: US 2015/0047651 A1
(43) Pub. Date: Feb. 19, 2015

(54) COMPACT HIGH FIDELITY EARPLUG

(71) Applicant: Etymotic Research, Inc., Elk Grove Village, IL (US)

(72) Inventors: Andy Haapapuro, Arlington Heights, IL (US); Viorel Drambarean, Lincolnwood, IL (US); Mead C. Killion, Elk Grove Village, IL (US); Gail Gudmundsen, Elk Grove Village, IL (US); Jonathan Stewart, Bloomingdale, IL (US)

(73) Assignee: Etymotic Research, Inc., Elk Grove Village, IL (US)

(21) Appl. No.: 14/271,972

(22) Filed: May 7, 2014

Related U.S. Application Data
(60) Provisional application No. 61/866,299, filed on Aug. 15, 2013.

(51) Int. Cl. A61F 11/12

(52) U.S. Cl. CPC A61F 11/12; A61F 2011/085
USPC 128/866; 128/867

(57) ABSTRACT

The present technology relates to a compact high fidelity earplug. The earplug has a central column having a hollow sound channel. Inner and outer platforms extend radially from the central column to define a first and second chamber. Openings in the first and second platforms allow sound to enter into the first chamber, and pass into the second chamber. Sound is then directed through an opening in the central column, into the sound chamber, and towards the ear of a wearer.

### RELATED APPLICATIONS

This application makes reference to, and claims priority to U.S. Provisional Patent Application No. 61/866,299 filed on Aug. 15, 2013, titled “Compact High Fidelity Earplug.” U.S. Non-Provisional Patent Application No. 61/866,299 is hereby incorporated by reference in its entirety.

### BACKGROUND

Repeated or prolonged exposure to sounds of a sufficiently high sound pressure level (SPL) can cause temporary or permanent hearing loss. Earplugs and/or earmuffs are available instruments that can help reduce the sound pressure level entering a wearer's ear, thereby limiting and/or reducing the likelihood of hearing loss in the wearer. Earplugs and earmuffs, however, can have shortcomings that make them less than ideal for use in certain situations. For example, earplugs and/or earmuffs can provide uneven or inconsistent attenuation across frequencies, such that a user can be inhibited or prevented from hearing certain types sounds or noises that they may otherwise wish to hear.

U.S. Pat. No. 5,113,967, issued to Killion et al. (hereinafter, “the '967 patent,” which is hereby incorporated by reference in its entirety) describes a relatively inexpensive, high-fidelity ready-to-wear earplug called the ER20 HiFi Earplug (hereinafter, “the ER20 earplug”). The ER20 earplug uses damping combined with the length resonance of the sound channel to provide hearing protection from exposure to sounds that are intense enough to risk hearing damage or discomfort. When combined with an eartip (e.g., the E-A-R triple-flange eartip), the ER20 earplug provides real-ear attenuation within about 3 dB of the desired 20 dB attenuation target for audio frequencies ranging from 250 to 6000 Hz. The ER20 earplug has been adopted by musicians and workers that desire to protect their ears by attenuating sound without inhibiting their ability to appreciate the various qualities of the music or other sounds. In order to achieve the desired attenuation and sound quality, the ER20 earplug incorporates a “re-entry horn” design that results in a longer earplug length. That is, the re-entry horn of the ER20 earplug can be of a length that extends beyond the edge of a wearer's ear. However, an earplug that extends out of the ear can be difficult to hide, and therefore may be aesthetically or cosmetically displeasing. Moreover, a long earplug can be uncomfortable for certain users, for example, users that may be wearing a hat, helmet, or earmuffs that cover the wearer's ears.

### SUMMARY

The present technology provides an earplug, for example, a compact, high fidelity earplug. In certain aspects, the earplug includes a central column extending from an inner column tip to an outer column face. The central column can have a hollow sound channel and a column opening. In some embodiments, the earplug also has an inner platform extending radially from the central column. The inner platform can comprise one or more inner platform openings. The earplug can also include an outer platform extending radially from the central column. The outer platform can have one or more one outer platform openings. The earplug can also include a cap having an outer end. The inner platform and the outer platform define a first chamber, and the outer platform and the exterior end of the cap define a second chamber. In some embodiments, the inner platform opening(s) is configured to allow external sound to enter into the first chamber, and each outer platform opening is configured to allow sound from the first chamber to enter into the second chamber, and the column opening is configured to allow sound from the second chamber to enter into the sound channel. In some embodiments, the sound channel is configured to allow sound to flow through the sound channel towards the inner column tip.

Certain aspects of the present disclosure provide an earplug for insertion into an ear of a wearer. In some embodiments, the earplug has an inner end and an outer end, the outer end extending away from the ear of a wearer when the earplug is inserted in the ear. The earplug can have a first chamber with a first opening, the first chamber positioned between the inner end and the outer end of the earplug. The first chamber can be adapted to receive external sound through the first opening. The earplug can also include a second chamber with a second opening. The second chamber can be positioned between the first chamber and the outer end and adapted to receive sound from the first chamber through the second opening. The earplug can also include a central column extending from the inner end towards the outer end along a longitudinal axis of the earplug. The central column can include a hollow sound channel and a third opening allowing sound from the second chamber to enter into the sound channel. In some aspects, the earplug directs sound through the sound channel towards the ear of the wearer.

The present technology also presents methods of attenuating sound within the ear canal. In certain embodiments, the method includes inserting an earplug into an ear canal of a wearer. The earplug can be one of the earplugs described in accordance with one or more embodiments of the present disclosure. For example, the earplug can have an inner end that faces the ear of the wearer, and an exterior end that faces away from the ear of the wearer. In certain aspects, the method includes directing external sound into a first chamber of the earplug, directing sound from the first chamber away from the ear of the wearer into a second chamber of the earplug, and directing sound from the second chamber, into a sound channel, and towards the ear of the wearer.

Certain embodiments of the present disclosure also describe an earplug assembly. The earplug assembly can comprise a first and second earplug, and a connector cord connecting the earplugs. In some embodiments, the connector cord comprises a first strand attached to the first earplug at a plug end of the strand and a second strand attached to the second earplug at a plug end of the strand. The connector cord can also include a first adjustment slider attached to a slider end of the first strand. The first adjustment slider can have at least one through hole. The connector cord can also include a second adjustment slider attached to a slider end of the second strand. The second adjustment slider can also have at least one through hole. In some embodiments, the first strand passes through and is freely slideable within a through hole of the second adjustment slider. In some aspects of the present technology, the second strand passes through and is freely slideable within a through hole of the first adjustment slider.

Some embodiments of the present technology provide an earplug assembly comprising two earplugs. In some embodiments, each earplug includes a central column extending from an inner column tip to an outer column face. The central column can have a hollow sound channel and a column opening. In some embodiments, the earplug also has an inner platform extending radially from the central column. The inner platform can comprise one or more inner platform openings. The earplug can also include an outer platform extending radially from the central column. The outer platform can have one or more one outer platform openings. The earplug can also include a cap having an outer end. In some embodiments, the earplug assembly also includes a connector cord connecting the earplugs. The connector cord can have a first strand attached to the first earplug at a plug end of the strand and a second strand attached to the second earplug at a plug end of the strand. The connector cord can also include a first adjustment slider attached to a slider end of the first strand. The first adjustment slider can have at least one through hole. The connector cord can also include a second adjustment slider attached to a slider end of the second strand. The second adjustment slider can also have at least one through hole. In some embodiments, the first strand passes through and is freely slideable within a through hole of the second adjustment slider. In some aspects of the present technology, the second strand passes through and is freely slideable within a through hole of the first adjustment slider.

### BRIEF DESCRIPTION OF SEVERAL VIEWS OF THE DRAWINGS

FIG. 1 is a side view of a compact high fidelity earplug in accordance with at least one embodiment of the present technology.

FIG. 2 is a cross-sectional side view of the earplug of FIG. 1.

FIG. 3 is a cross-section of a side view of an earplug of the present technology without an eartip.

FIG. 4 is another side view of an earplug of the present technology without an eartip, showing internal structure of the earplug in broken lines.

FIG. 5A is a three-dimensional isometric view of an earplug of the present technology with the cap removed, and without an eartip.

FIG. 5B is a three-dimensional isometric view of an earplug of the present technology with the cap removed, showing the direction of flow of sound throughout the earplug in accordance the present technology.

FIG. 6 is a side view of an earplug of the present technology without a cap and eartip, and that shows internal structure of the earplug in broken lines.

FIG. 7 illustrates an exterior side view of an earplug of the present technology compared with an ER20 earplug.

FIG. 8 is a chart showing the frequency response for an ER20 measured in a production test fixture.

FIG. 9 is a chart showing the frequency response for an earplug of the present technology measured in a production test fixture.

FIG. 10 is a chart showing the real-ear response of the open ear, the ER20 earplug, and an earplug of the present technology, measured across multiple frequencies on a KEMAR Manikan with an ear-simulator coupler.

FIG. 11 shows an earplug assembly including a connector cord in accordance with at least one embodiment of the present technology.

FIG. 12 shows another configuration of an earplug assembly with a connector cord in accordance with at least one embodiment of the present technology.

FIG. 13 shows a close up view of an adjustment slider used in connection with a connector cord in accordance with at least one embodiment of the present technology.

FIG. 14 illustrates a flow diagram of a method in accordance with at least one embodiment of the present technology.

### DETAILED DESCRIPTION

The present technology relates to high fidelity earplugs. More specifically, the present disclosure describes compact earplugs that provide approximately uniform sound attenuation across a wide array of frequencies. Even more particularly, the present technology describes ready-to-wear earplugs that are more compact (i.e., shorter in length) than previous high fidelity length resonance earplugs, thereby providing aesthetic, functional, and other practical benefits.

The present technology provides an earplug that is shorter than the ER20 earplug, while offering similar or improved attenuation properties. The earplugs of the present technology can be used with a 3-flange eartip, for example, the 3 flange eartips described in the '967 patent. The present technology also implements similar mechanical and/or acoustical properties of the sound-conducting stem described in the '967 patent, which can be designed to operate effectively with the 3-flange eartips. The present technology provides these features while offering an earplug that is shorter in length. As such, the presently described earplugs extend to a lesser amount out (or even not at all) out of the ear of a wearer. The present technology also provides systems and methods for producing earplugs at a lower cost than for that of the ER20 earplugs.

The present technology also provides a connector cord that allows for two earplugs to be connected together for wearing and storage.

As described herein, the ER20 earplug described in the '967 patent uses damping techniques and length resonance to provide sound attenuation that offers hearing protection for wearers, without inhibiting the quality of sound delivered into the ear canal. The ER20 earplug reduces the pressure level of sound entering the wearer's ear, but it provides a relatively uniform attenuation across a wide range of sound frequencies. That is, the ER20 earplug allows a wearer to reduce the level of sound entering the ear canal relatively evenly across a wide spectrum of sound frequency or pitch levels. In this manner, a wearer can be able to provide protection to the ears without having to struggle to perceive sounds at a certain frequencies.

For example, factory workers may have previously been forced to choose between providing no sound attenuation (i.e., wearing no ear protection), or providing too much sound attenuation while risking the chance of missing out on important factory sounds, such as warning signals, improperly operating equipment, approaching vehicles, or talking co-workers. Prior to the ER20, such a worker may have considered using an attenuator such as earmuff drilled with one or more holes, or a foam earplug only partially inserted in the ear. The relatively uniform attenuation provided by the ER20 earplug helped alleviate this problem

The ER20 earplugs, however, have a relatively long length. In order to produce the relatively uniform and accurate attenuation, the ER20 earplug operates in a manner that seeks to replicate the normal external-ear resonance of the ear canal, concha, and pinna at an attenuated level. This external-ear resonance occurs at about 2.7 kHz in an average, normal ear.

To replicate this resonance, the ER20 earplugs incorporate a “re-entry horn” feature. The re-entry horn of the ER20 provides a resonance that can be calculated by the formula f=86/L, where L is measured in millimeters (mm) and f is provided in kHz. In order to provide a quarter-wave resonance of about a 2.7 kHz (thereby replicating the resonance of a normal ear), the ER20 earplug utilizes a re-entry horn that is approximately 32 mm in folded length (that is, 2.7 kHz≈86/32 mm). This folded length allows for the earplug to be more compact than if the re-entry horn were an additional 32 mm in length.

In certain situations, however, even this 32 mm re-entry horn can be cumbersome, uncomfortable, and/or not aesthetic or cosmetic. For example, a user that desires to wear a helmet (e.g., a motorcycle helmet) may be unable to wear an ER20 earplug, because the 32 mm re-entry horn can extend beyond the ear, thereby resulting in uncomfortable and/or painful pressure from a tight-fitting helmet.

Additionally, certain users may wish to achieve the same level of high fidelity attenuation, without appearing as though they are wearing earplugs. The re-entry horn of the 32 mm long ER20 earplug can make this difficult, as it is likely to extend beyond the plane of the concha-pinna of the ear, and therefore appear visible to observers.

The present technology provides advantages by providing an earplug that uses two circular chambers or “apartments” in a base section of the earplug. The earplugs of the present technology allow for sound to enter a first chamber through an opening below an inner platform, or “first floor.”

The present technology then directs that sound through one of several openings to a second chamber, defined by the first platform and a second platform, or “second floor.” Once in the second chamber, the sound is directed to flow around the periphery of the second chamber to another opening, or an entrance to a sound channel, or “empty enclosed elevator shaft,” which runs down a central column of the earplug.

The sound channel runs back through the earplug, past the second chamber and the first chamber, then through an acoustic resistance member, and then down a sound-conducting stem into the eartip where it is delivered into the ear canal. By using this round chamber, or “circular apartment” approach as opposed to a re-entry horn approach, the present technology provides an earplug that can be about 6 mm shorter in length overall compared with the ER20 earplug. This reduction in overall length can be sufficient to move the external tip of the earplug inside the plane of the concha-pinna of the ear for many users. This approach offers similar or improved the attenuation frequency response and attenuation characteristics when compared with the longer ER-20 earplugs.

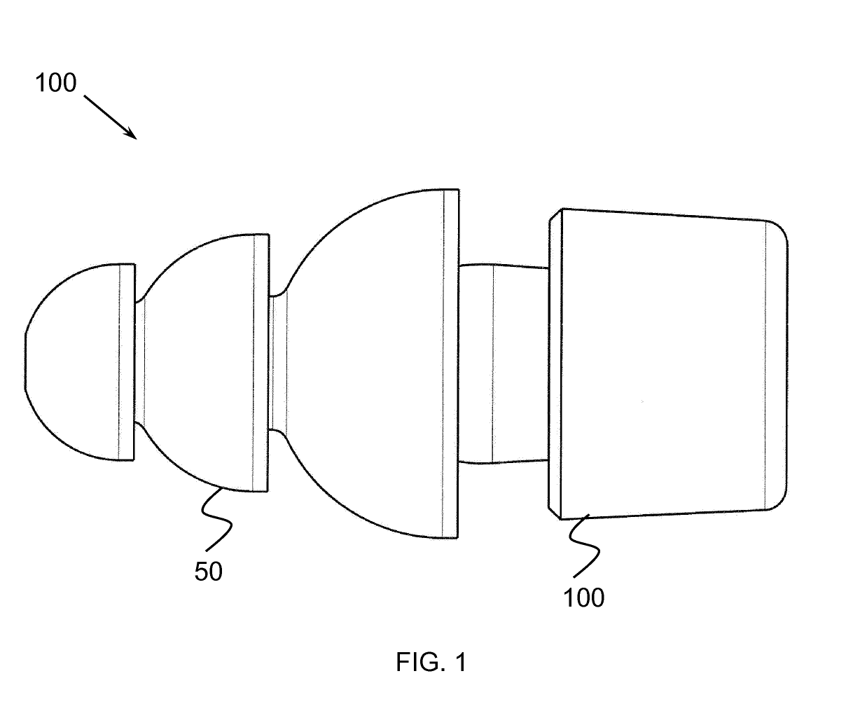

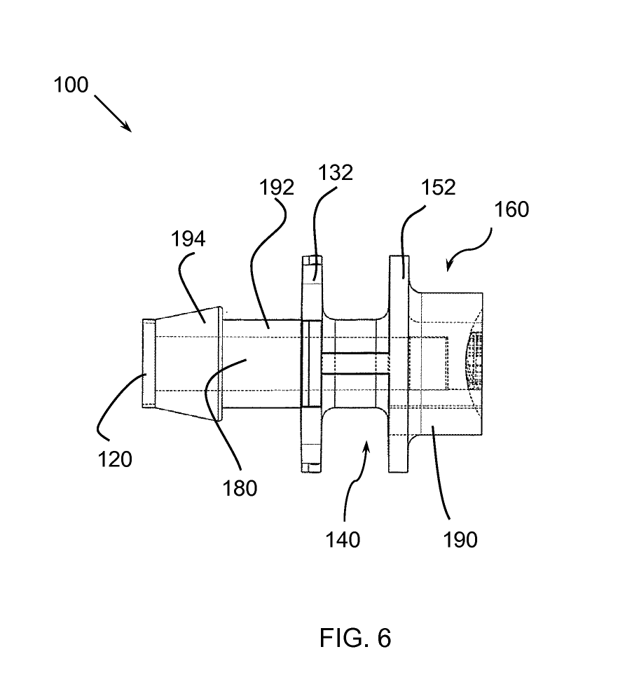

FIGS. 1-11 illustrate various embodiments of the present technology, and chart the functionality of the present technology with respect to other earplug designs. For example, FIG. 1 provides a side view of a compact high fidelity earplug 10, which includes a base portion 100, and an eartip 50.

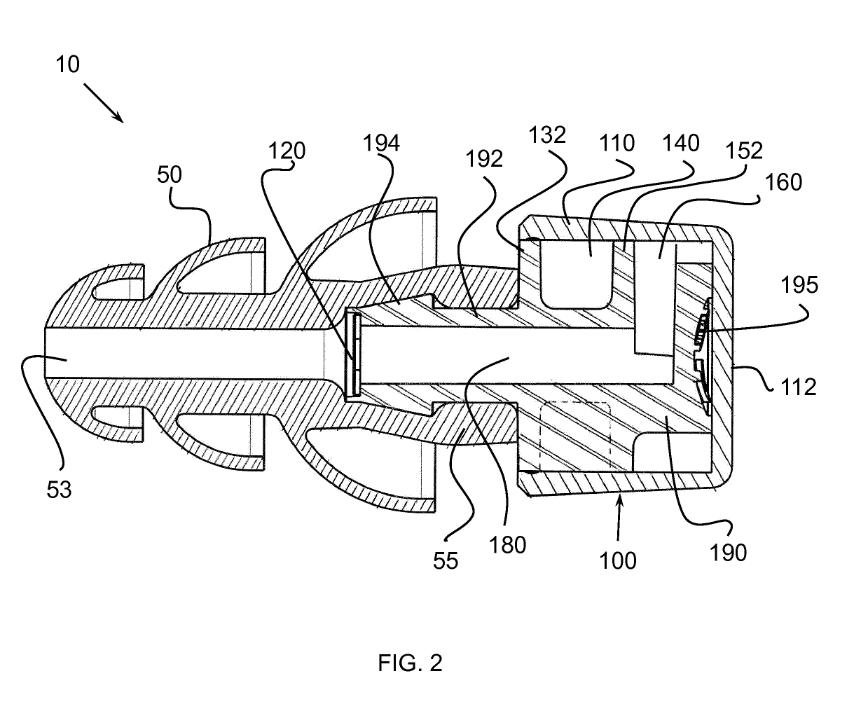

FIG. 2 shows the internal structure of an earplug 10 having an eartip 50 and base section 100. The eartip 50 can be configured to sit at a floor of a concha of a wearer's ear, and can take many forms or shapes. For example, the eartip 50 can be the tri-flange eartip used in connection with the ER-20 earplugs and described in the '967 patent. Other eartips, such as foam eartips, could also be used provided that the overall length of the sound channel in the eartip is preserved. As shown in FIG. 2, eartip 50 includes a channel 53, which, in combination with the sound channels 160 and 190, provides an auxiliary quarter-wave resonance boost to improve high-frequency performance at frequency levels above 2.7 kHz.

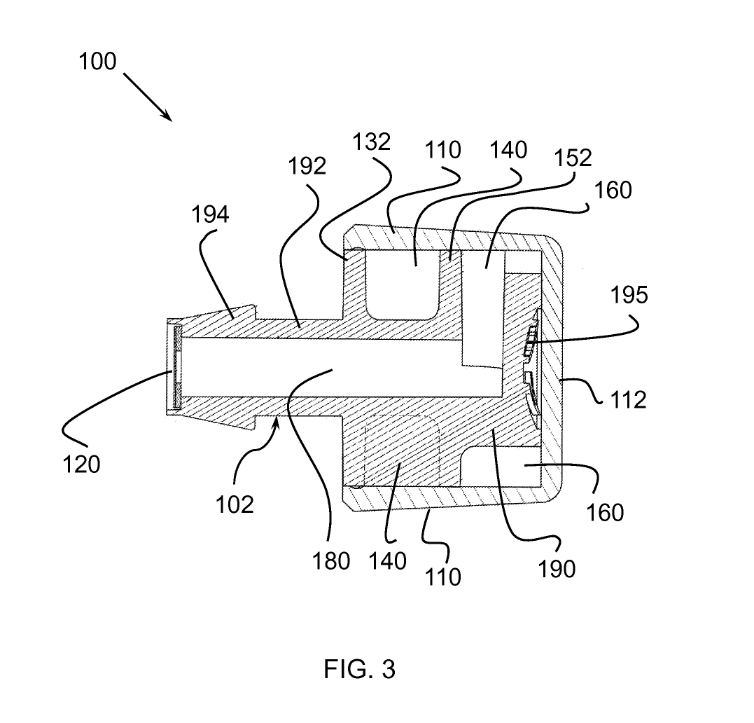

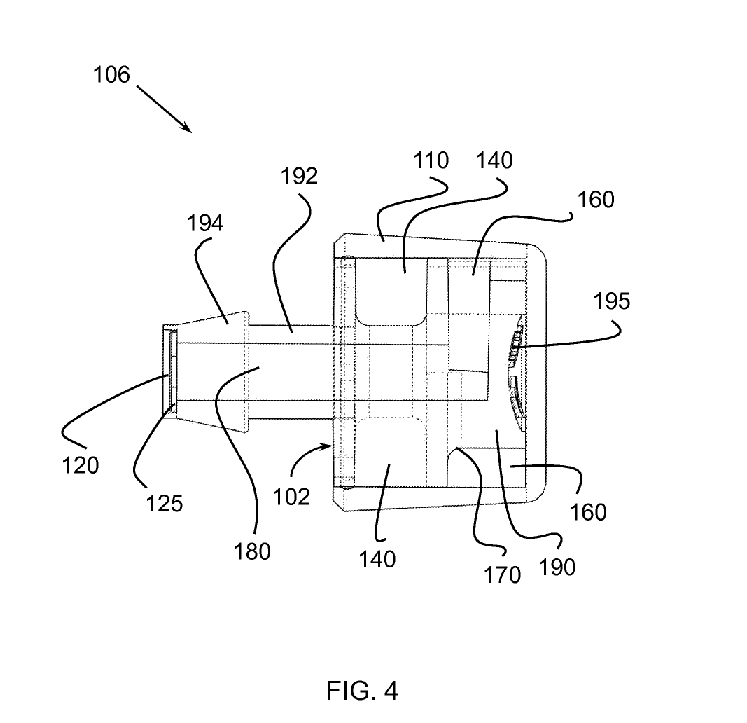

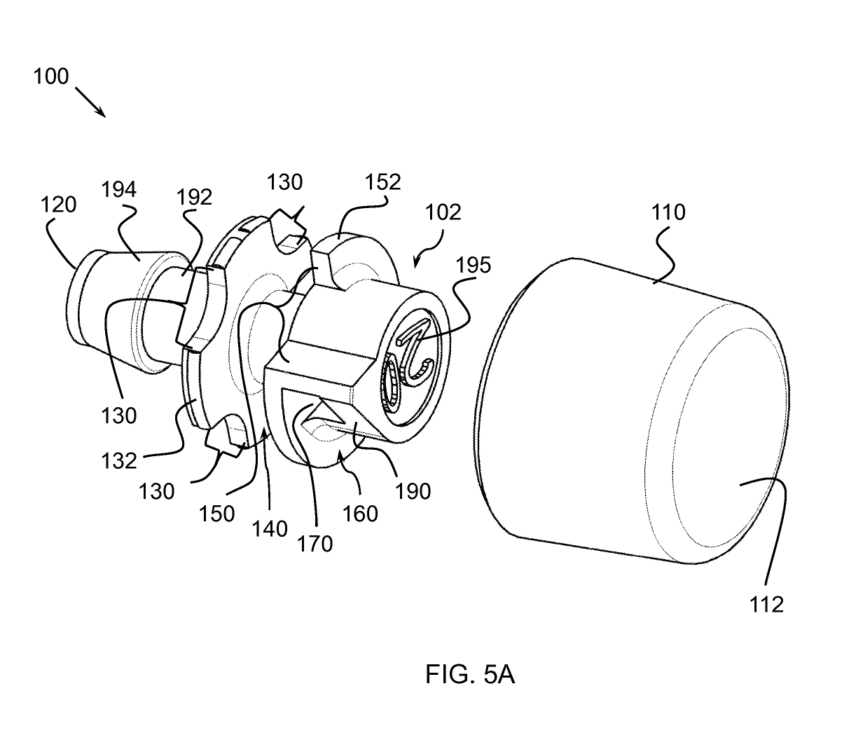

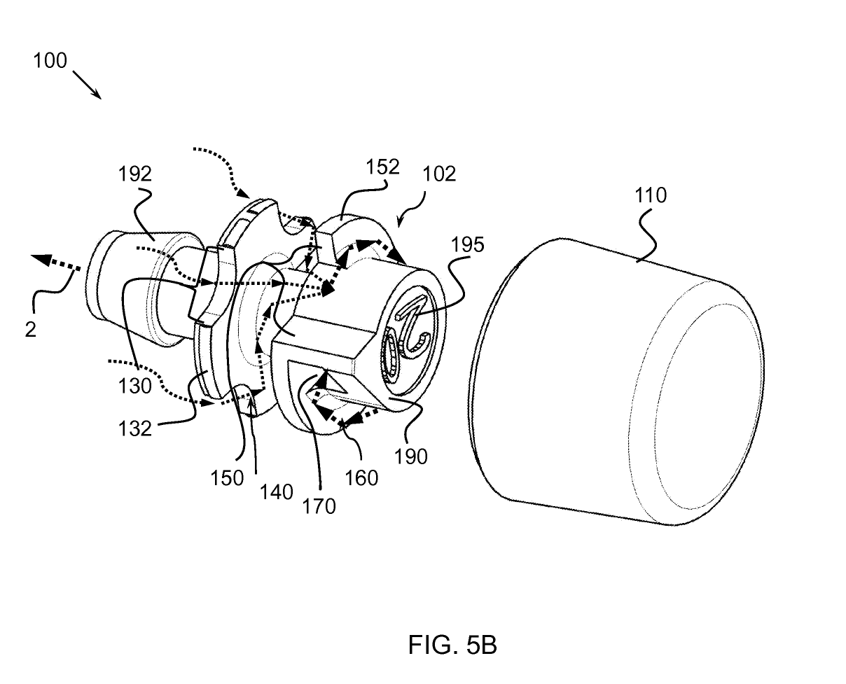

FIGS. 3, 4, 5A, 5B, and 6 provide various views of an earplug 10 of the present technology. More specifically, FIG. 3 depicts a cross-section of an earplug 10 without an eartip. FIG. 4 shows another view of the base section 100 of the earplug 10 without the eartip 50, and depicts the exterior of the base section 100 with the internal structure represented in broken lines. FIGS. 5A and 5B provide isometric view of the earplug without an eartip, and with the cap removed.

As shown in FIGS. 2-5B, the base section 100 of the earplug comprises a cap 110, which in certain embodiments, can be removable from the base section 100 and the earplug 10. The outer end of the earplug 112 (or the exterior end of the cap) is at a point furthest from the eartip 50. In use, the outer end 112 sits at the most external edge to the earplug 10 when inserted into the ear of a wearer.

As seen in FIGS. 2-5B, the base section 100 of the earplug 10 has a first platform 132 (or an inner platform) that includes one or more openings 130. The openings 130 allow external sound to enter the earplug into a first chamber 140. The first chamber 140 is defined by the first platform 132 and a second platform 152 (or an outer platform).

The outer platform 152, which also includes an outer platform opening 150. The outer platform opening 150 allows sound from the first chamber 140 to enter into a second chamber 160. The second chamber 160 includes a portion of a central column 190, which extends through the center of the base section along the longitudinal axis of the earplug. The size of the first and second chamber can vary. For example, in certain embodiments, the first chamber can have an inner diameter of about 0.136″, an outer diameter of about 0.336″, and a depth of about 0.107″, and a volume of about 0.008 cubic inches. In certain embodiments, the second chamber can have an inner diameter of about 0.220″, an outer diameter of about 0.336″, a depth of about 0.116″, and a volume of about 0.006 cubic inches.

The central column 190 extends from an inner column tip 120 towards an outer column face 195, which can be adjacent to the exterior end 112 of the earplug 10. In some embodiments, the outer face 195 of the central column can comprise symbols, letter, or numbers to identify certain functionality of the earplug. For example, in FIGS. 5A and 5B, the exterior end 195 is shown depicting the number “20,” which can represent that the earplug provides 20 dB of sound attenuation. The shape of outer face end 195 can also be used to provide structural support for the earplug, or the cap, for example.

A hollow sound channel 180 runs through the central column 190 along the longitudinal axis of the column. In some aspects, the central column 190 has a stem portion 192 that is configured to connect with an eartip 50. The stem 192 can comprise a flange or a collar 194 that provides a friction-tight fit for installation of a flexible eartip 50. For example, the central column 190 can be configured so that a flexible eartip 50 (as shown in FIGS. 1 and 2) with a flexible exterior tube section 55 can slide over the step 192 and collar 194. As shown in FIG. 1, the collar 194 is angled to increase the outer diameter of the stem 192, and therefore grip the exterior tube section 55 of the eartip.

The central column 190 can also comprise a third opening 170, which allows sound from the second chamber 160 to enter into the sound channel 180. In certain embodiments, the third opening 170 is positioned such that sound entering the second chamber 160 is directed around a majority of the central column 190 before entering into the sound channel 180 of the central column.

FIG. 5B provides a depiction of the flow path 2 of sound within the base section of the earplug. As shown, sound from the exterior enters into the first chamber 140 of the base section 100 through an opening 130 (or multiple openings) in the first platform 132 of the earplug. Sound from the first chamber 140 then enters into the second chamber 160, through an opening 150 in the second platform 152.

In certain embodiments, the cap 110 and the exterior end 112 of the cap 110 redirects the sound back towards the ear of a wearer. That is, the sound is then directed around the circumference of the central column 190 into the third opening 170 of the central column. The second opening and the third opening can be positioned such that the sound must travel 180 degrees or more around the central column 180 before reaching the third opening 170. The openings may be positioned such that sound must travel 270 degrees or more around the central column 190 before reaching the third opening 170.

Sound entering the third opening then proceeds through the sound channel (shown as 180 in FIGS. 2-4 and 6), and is directed towards the ear of the wearer, through the stem 192 of the central column 190. The sound channel 180, therefore, runs through the base section 100, past the second chamber 160 and the first chamber 140.

In some aspects, the sound channel can comprise an acoustic resistance member 125 (see FIG. 4). The acoustic resistance member can help smooth the acoustic response, for example, so that the acoustic response corresponds to that of a natural open ear before delivering sound into the ear canal of the wearer.

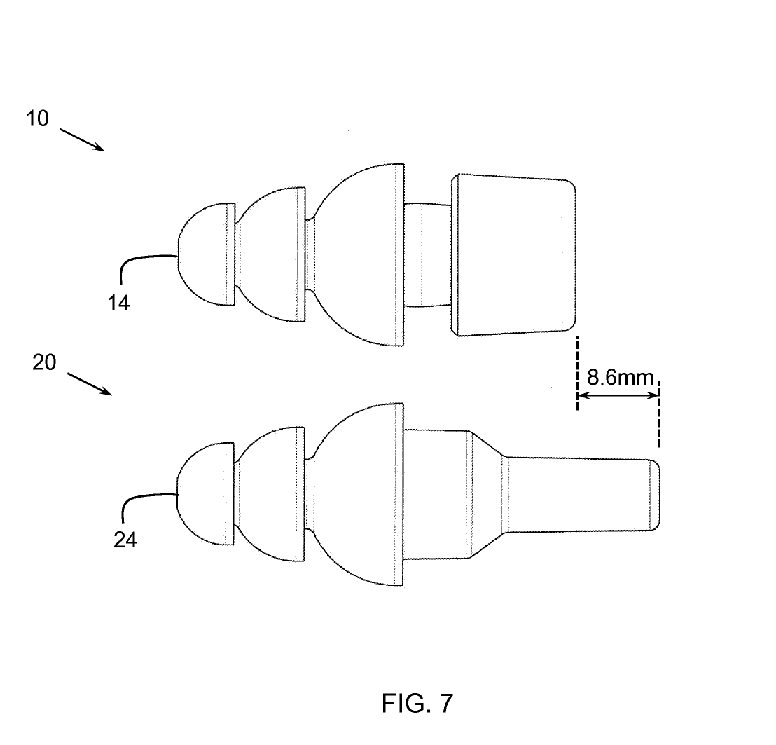

FIG. 7 illustrates an exterior side view of an earplug of the present technology 10 compared with an ER20 earplug 20. FIG. 7 demonstrates that there is a significant reduction in length between the ER20 earplug 20 and the earplug 10 of the present technology. As shown, when the interior tips (14 and 24) of the earplugs (10 and 20) are aligned, the earplug of the present technology 10 is significantly shorter in length than the ER20 earplug 10. By using this round chamber, or “circular apartment” approach, the present technology provides an earplug that can be about 8.6 mm shorter in length overall compared with the ER20 earplug. This reduction in overall length can be sufficient to move the external tip of the earplug inside the plane of the concha-pinna of the ear for many users. This approach offers a similar or improved sound attenuation response and characteristics when compared with the longer ER-20 earplugs.

The dimensions of the central column 190 and the sound channel 180 and components of the earplug can vary depending on the length of the earplug, and the value of the attenuation. For example, in certain embodiments, the sound channel 180 can have an inner diameter of about 0.080″, and a length of about 0.450″. In certain aspects, the inner diameter of the sound channel can be about 1.5 times smaller than the inner diameter of the first chamber 140.

In use, the base section 100 of the earplug sits at or around the floor of the concha of a wearer's ear. The sound pressure at that the floor of the concha of an ear is typically increased relative to the sound arriving at the ear as a result of the resonances of the concha and pinna of the ear. Accordingly, the present technology reduces this sound pressure by directing sound from around the base of the earplug thru the openings (e.g., 130 and 150), into the first and second chambers (e.g., 140 and 160), around the central column 190, into the sound channel 180 and towards the ear. This redirection of sound results in a reduction in sound pressure level entering the ear canal, while maintaining a relatively high sound quality and clarity across a broad range of sound frequencies.

In certain embodiments of the present technology, the earplug 10 can comprise a pull string or a pull cord. The pull cord can be used, for example, to facilitate removal of the earplug from the ear. The pull cord can have particular value with the earplug of the present technology, which, depending on individual fit, may be difficult for a user to grasp for removal due to the shortened length.

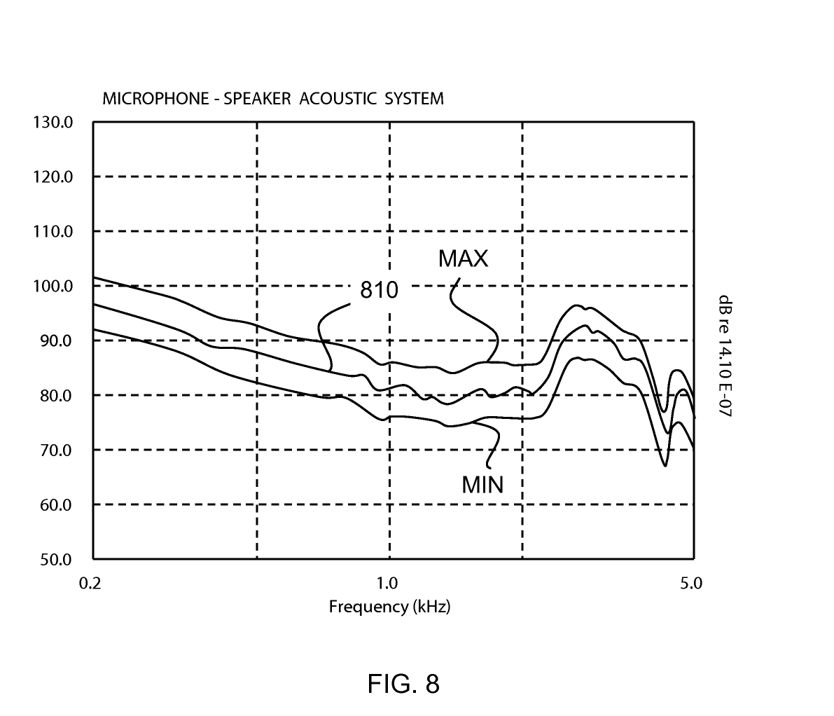

FIG. 8 is a chart showing the frequency response 810 for an ER20 measured in a production test fixture, along with the maximum and minimum limits values. That is, curve 810 represents the response of the previous earplug when compared to the response of an earplug of the present technology. The chart demonstrates the results from tests that establish the repeated acoustic performance of the present technology.

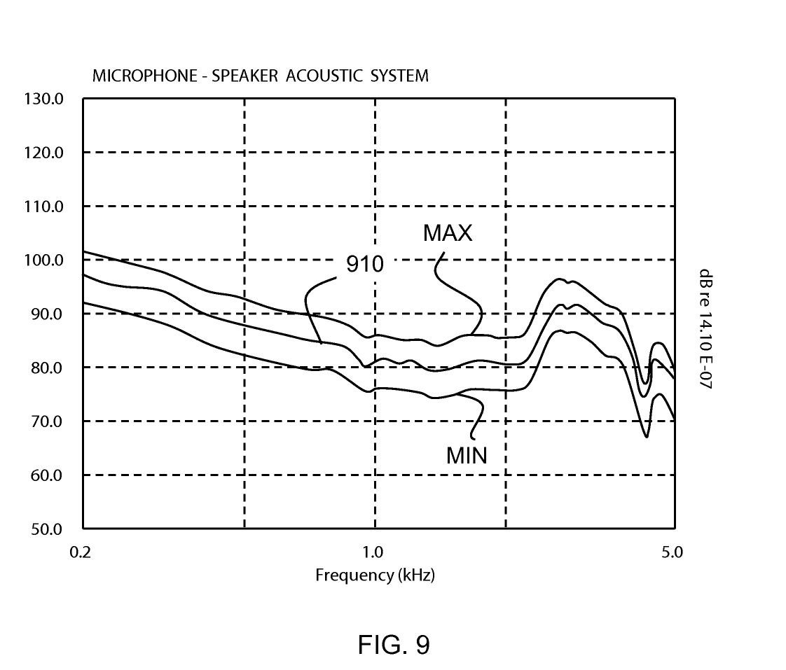

Similarly, FIG. 9 is a chart showing the frequency response 910 for an earplug of the present technology measured in a production test fixture along with the maximum and minimum response values. As shown in FIGS. 8 and 9, the frequency response for each of the ER20 earplug, and the earplug of the present technology are centered within the MAX/MIN limits, and show essentially the same response in that test fixture. Accordingly, the earplugs of the present technology provide the same or improved frequency response as the ER20 earplug, in a shorter, more compact, and convenient design.

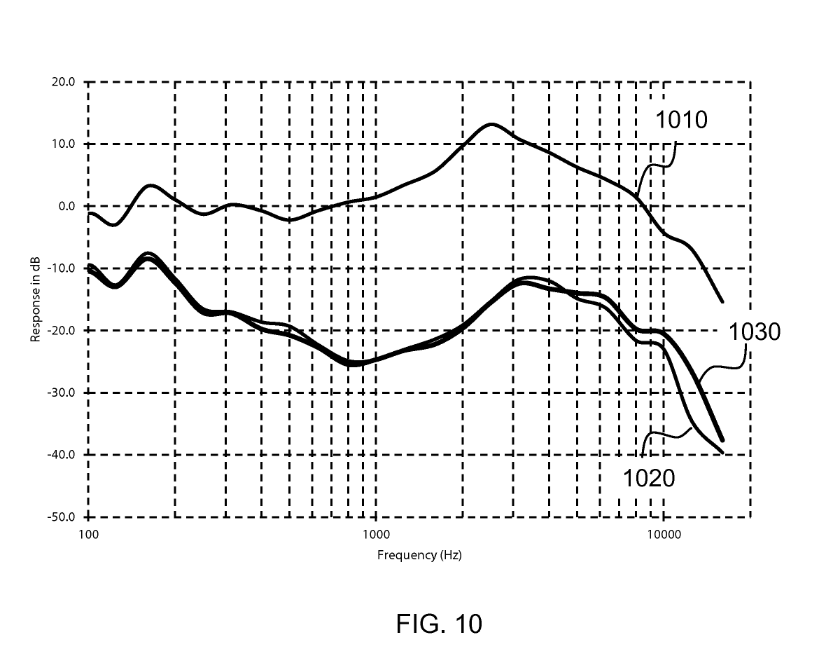

Tests were conducted to record the response to sounds of various frequencies in an open ear, an ear protected with the ER20 earplug, and an ear protected with an earplug of the present technology. The tests used a KEMAR manikin, which is an acoustic manikin with structural features representing the anatomical dimensions an average person. In particular, the KEMAR manikin represents a person with an ear containing acoustic chambers that, in total, provide a similar, or essentially the same acoustic load as an average normal human ear. (For more specifics about the manikin measurements, see: Proceedings of a conference; MD Burkhard 1978, republished by G.R.A.S.) FIG. 10 is a chart showing response of these tests.

More specifically, FIG. 10 shows the response of a KEMAR manikin to a uniform diffuse sound fields with: (1) an open ear (curve 1010); (2) the ER20 earplug (curve 1020); and (3) the earplug of the present technology (curve 1030). The chart demonstrates the resonance effects of the ear canal, concha, and pinna, which are notably present at frequencies at and above about 2.7 kHz. As demonstrated by the graph of FIG. 10, curves 1020 and 1030 show a similar response, consistent with the test data demonstrated in FIGS. 8 and 9. Additionally, for frequencies above 10 kHz, the present technology provides slightly less attenuation than the ER20 earplug, which can be desirable, because higher pitch frequencies are more difficult.

The present technology also provides an adjustment slider for a connector cord. For example, a connector cord can be used to connect two earplugs such that the earplugs can remain connected when not in use. The connector cord can be designed to be placed over the neck and/or shoulders of a wearer when not in use. The connector cord can also be of an adjustable length, so that a wearer can increase or decrease the distance between the connected earplugs, for example, to accommodate for different head and/or ear sizes of different users.

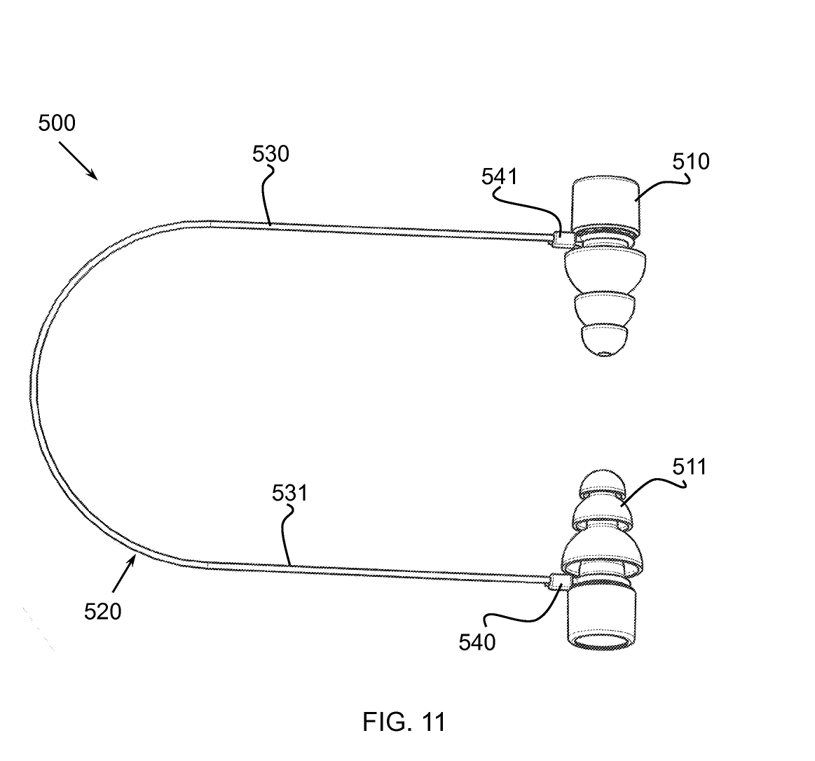

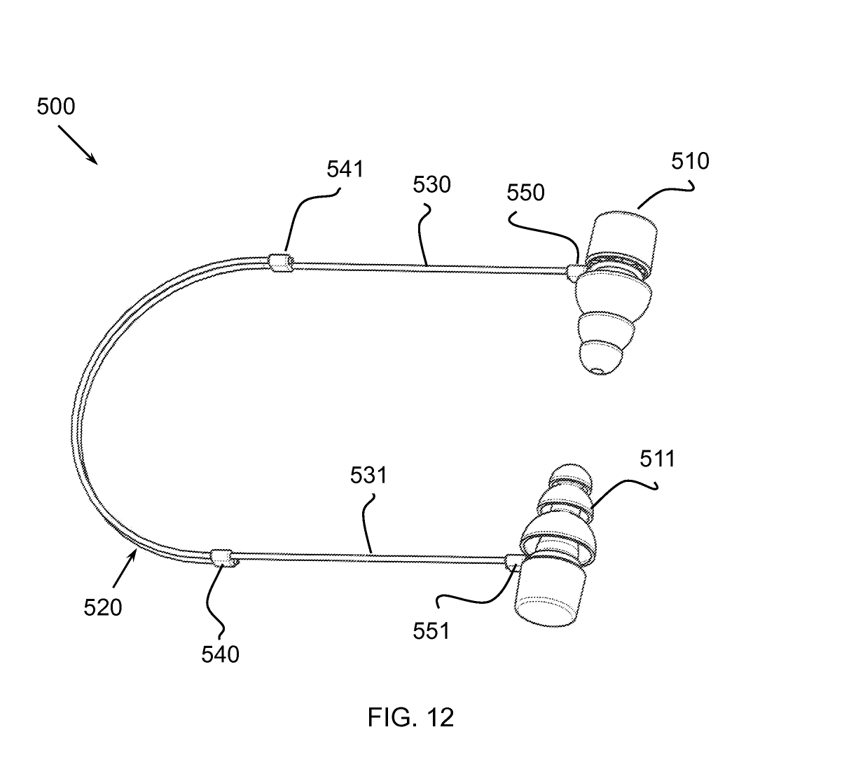

FIGS. 11 and 12 show configurations of an earplug assembly 500 including a connector cord 520 in accordance with at least one embodiment of the present technology. The earplug assembly 500 comprises two earplugs 510 and 511, which can be, for example, the compact, high fidelity earplugs of the present technology. The earplugs 510 and 511 are connected by a connector cord 520, which can be used to keep a pair of earplugs together, for example, so that one earplug of a set will not be separated from the other.

FIG. 11 shows the earplug assembly 500 in a compressed position, while FIG. 12 shows the assembly 500 in an extended, or lengthened position. As shown in these figures, the connector cord 520 comprises two strands 530 and 531, and two adjustment sliders 540 and 541. As seen in FIG. 12, strand 530 is attached to earplug 510 at a connection point 550. Likewise, strand 531 is attached to earplug 511 at a connection point 551. The strand 530 passes through a through hole of adjustment slider 541, and is connected to adjustment slider 540 at the opposite end of the strand 530. Likewise, strand 531 passes through a through hole of adjustment slider 540, and is attached to adjustment slider 541 at the opposite end of the strand.

In certain embodiments, the cord 520 material can be a stretchable cord, for example, using a product like Stretch® Magic. The stretchable cord allows the strand to flex and stretch in an elastic manner, without breaking or tearing, for example. The cord 520 can vary in thickness, for example, the cord can be approximately 0.7 mm to 1.5 mm in thickness.

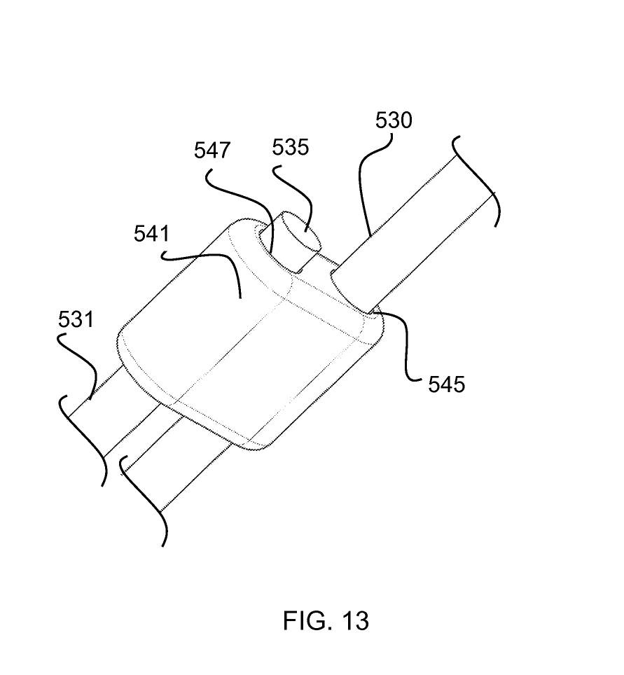

FIG. 13 illustrates a close up view of an adjustment slider 541. The adjustment slider 540 can comprise a first through hole 545 and a second through hole 547. A strand 530 can then be placed through one through hole (e.g., hole 545 as shown in FIG. 13) and routed to another earplug. Similarly, another strand 531, which is attached at one end to an earplug, can be adhered at the other end 535 adhered to the second through hole 547, via a solvent or adhesive. Strand 530 is allowed to slide freely in the first through hole, and strand 531 is allowed to slide freely through a through hole in another adjustment slider, for example, slider 540, as shown in FIGS. 11 and 12.

The user can increase or decrease the distance between the earplugs by sliding one or both ends of the strands 530 and 531 through the adjustment sliders 540 and 541, thereby increasing or decreasing the overall length of the cord 520. For example, to lengthen the cord 520, a user can grasp the cord 520 at either end and pull to a desired length. Conversely, to shorten the cord 520, the user can pull the adjustment sliders 540 and 541 apart from one another.

FIG. 11 shows the earplug assembly 500 in a shortened position, with each adjustment slider 540 and 541 in contact, or in very near position to the earplugs 511 and 510, respectively. FIG. 12, on the other hand, shows the earplug assembly 500 in a slightly extended position, with the adjustment sliders 540 and 541 in a position along strands 531 and 530 away from the earplugs 511 and 510. In some embodiments, the cord 520 can have a fully extended length that is approximately twice as long as the fully shortened length.

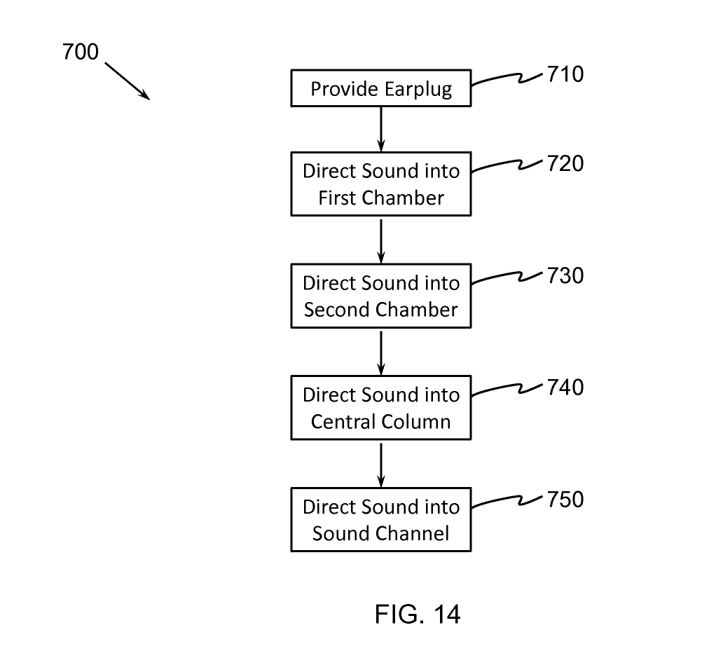

The present technology also provides methods of attenuating sound within the ear canal. FIG. 14 illustrates a flow diagram of a method 700 in accordance with at least one embodiment of the present technology. In certain embodiments, method 700 can include the step 710 of providing an earplug (e.g., earplug 10 described above with respect to FIGS. 1-7) for insertion into an ear canal of a wearer. The earplug can have an inner that faces the ear of the wearer, and an exterior end that faces away from the ear of the wearer.

At step 720, sound is directed into a first chamber of the earplug, for example, through one or more openings in a platform of the earplug. The first chamber can be defined, for example, by the first platform and a second platform.

At step 730, sound is directed from the first chamber into a second chamber of the earplug. For example, the sound can be directed from the first chamber, away from the ear of the wearer, through a second opening in the second platform.

At step 740, sound is directed around a central column of the earplug within the second chamber. In certain embodiments, the second chamber is defined by a cap and the second platform. The central column can have, for example, an opening or an entrance, which leads to the hollow interior portion of the central column, or a sound channel. In certain embodiments, sound is directed at least 180 degrees around the central column of the earplug before directing the sound into the third opening.

At step 750, sound is directed into the sound channel via the opening into the sound channel, and back towards the ear of the wearer. In certain embodiments, the sound is directed through the center or middle of the earplug, past the second chamber and first chamber, and through the center of a stem of the earplug. In certain embodiments, sound is then directed through an eartip, for example, a flexible triple-flange eartip, and into the ear canal of a wearer.

The present technology has now been described in such full, clear, concise and exact terms as to enable any person skilled in the art to which it pertains, to practice the same. It is to be understood that the foregoing describes preferred embodiments and examples of the present technology and that modifications may be made therein without departing from the spirit or scope of the invention as set forth in the claims. Moreover, while particular elements, embodiments and applications of the present technology have been shown and described, it will be understood, of course, that the present technology is not limited thereto since modifications can be made by those skilled in the art without departing from the scope of the present disclosure, particularly in light of the foregoing teachings and appended claims. Moreover, it is also understood that the embodiments shown in the drawings, if any, and as described above are merely for illustrative purposes and not intended to limit the scope of the invention, which is defined by the following claims as interpreted according to the principles of patent law, including the Doctrine of Equivalents. Further, all references cited herein are incorporated in their entirety.

1. An earplug configured to attenuate sound entering the ear of a wearer, the earplug comprising:
a central column extending from an inner column tip to an outer column face, the central column having a hollow sound channel and a column opening;
an inner platform extending radially from the central column, the inner platform comprising at least one inner platform opening;
an outer platform extending radially from the central column, the outer platform comprising an outer platform opening; and
a cap having an outer end;
wherein the inner platform and the outer platform define a first chamber, and the outer platform and the exterior end of the cap define a second chamber,
wherein each inner platform opening is configured to allow external sound to enter into the first chamber, and the platform opening is configured to allow sound from the first chamber to enter into the second chamber;
wherein the column opening is configured to allow sound from the second chamber to enter into the sound channel; and
wherein the sound channel is configured to allow sound to flow through the sound channel towards the inner column tip.

2. The earplug of claim 1, further comprising a flexible eartip configured to be inserted into the ear canal of a wearer.

3. The earplug of claim 2, wherein the eartip has an inner end, wherein the length of the earplug measured from inner end of the eartip to the outer end of the cap is less than about 1 inch.

4. The earplug of claim 2, wherein the central column comprises a collar extending radially from the central column, wherein the collar is configured to hold the eartip in place with respect to the earplug.

4. The earplug of claim 1, wherein the inner diameter of the first chamber is about 0.130 inches.

5. The earplug of claim 4, wherein the volume of the first chamber is about 0.008 cubic inches.

6. The earplug of claim 4, wherein the diameter of the sound channel is about 0.08 inches.

7. The earplug of claim 1, wherein the inner diameter of the first chamber is about 1.5 times greater than the inner diameter of the sound channel.

8. The earplug of claim 1, wherein the length of the earplug measured from the inner column tip to the exterior end of the cap is less than about 0.6 inches.

10. The earplug of claim 1, wherein the exterior end of the cap does not protrude beyond the plane of the concha-pinna when the earplug is inserted into the ear of a wearer.

11. The earplug of claim 1, further comprising a pull string, wherein the pull string facilitates removal of the earplug from wearer's ear.

12. The earplug of claim 1, wherein the inner platform comprises two or more inner platform openings.

13. The earplug of claim 1, wherein the earplug is configured so that sound entering the second chamber through the second opening travels more than about 180 degrees around the central column before entering the sound channel through the column opening.

14. An earplug for inserting into an ear of a wearer, the earplug comprising:
an inner end and an outer end, the outer end extending away from the ear of a wearer when the earplug is inserted in the ear;
a first chamber having a first opening, the first chamber positioned between the inner end and the outer end, the first chamber adapted to receive external sound through the first opening;
a second chamber having a second opening, the second chamber positioned between the first chamber and the outer end, the second chamber adapted to receive sound from the first chamber through the second opening;
a central column extending from the inner end towards the outer end along a longitudinal axis of the earplug, the central column comprising a hollow sound channel and a third opening allowing sound from the second chamber to enter into the sound channel;
wherein the earplug directs sound through the sound channel towards the ear of the wearer.

15. A method of attenuating sound within the ear canal comprising the steps of:
inserting an earplug into an ear canal of a wearer, the earplug having an inner end facing the ear of the wearer and an exterior end facing away from the ear of the wearer;
directing external sound into a first chamber of the earplug;
directing sound from the first chamber away from the ear of the wearer into a second chamber of the earplug;
directing sound from the second chamber, into a sound channel, and towards the ear of the wearer.

16. The method of claim 15, wherein the step of directing sound into the first chamber comprises directing sound through at least one first opening in an inner platform, the inner platform defining an inner boundary to the first chamber, and wherein the step of directing sound into the second chamber comprises directing sound through a second opening in an outer platform, the outer platform defining an outer boundary to the first chamber and an inner boundary to the second chamber.

17. The method of claim 16, wherein the step of directing sound into a sound channel comprises directing sound through a third opening in a central column of the earplug, wherein the sound channel represents a hollow interior portion of the central column.

18. The method of claim 17, wherein the step of directing sound into a sound channel further comprises directing sound at least 180 degrees around a central column of the earplug before directing the sound into the third opening.

19. An earplug assembly comprising:
a first earplug and a second earplug;
a connector cord connecting the first earplug with the second earplug, the connector cord comprising:
a first strand attached to the first earplug at a plug end of the strand;
a second strand attached to the second earplug at a plug end of the strand;
a first adjustment slider attached to a slider end of the first strand, the first adjustment slider having at least one through hole;
a second adjustment slider attached to a slider end of the second strand, the second adjustment slider having at least one through hole;
wherein the first strand passes through and is freely slideable within a through hole of the second adjustment slider, and wherein the second strand passes through and is freely slideable within a through hole of the first adjustment slider.

20. An earplug assembly comprising
a first earplug and a second earplug, each earplug comprising:
a central column extending from an inner column tip to an outer column face, the central column having a hollow sound channel and a column opening;
an inner platform extending radially from the central column, the inner platform comprising at least one inner platform opening;
an outer platform extending radially from the central column, the outer platform comprising an outer platform opening; and
a cap having an outer end;
a connector cord connecting the first earplug with the second earplug, the connector cord comprising:
a first strand attached to the first earplug at a plug end of the strand;
a second strand attached to the second earplug at a plug end of the strand;
a first adjustment slider attached to a slider end of the first strand, the first adjustment slider having at least one through hole;
a second adjustment slider attached to a slider end of the second strand, the second adjustment slider having at least one through hole;
wherein the first strand passes through and is freely slideable within a through hole of the second adjustment slider, and wherein the second strand passes through and is freely slideable within a through hole of the first adjustment slider.
